(19) United States
(12) Patent Application Publication
(10) Pub. No.: US 2017/0105710 A1
(43) Pub. Date: Apr. 20, 2017

(54) SYSTEM AND METHOD FOR EXTRACTING TISSUE SAMPLES

(71) Applicant: INDIANA UNIVERSITY RESEARCH AND TECHNOLOGY CORPORATION, Indianapolis, IN (US)

(72) Inventors: Luke G. Gutwein, Indianapolis, IN (US); Rajiv Sood, Carmel, IN (US); Sunil Tholpady, Carmel, IN (US)

(73) Assignee: INDIANA UNIVERSITY RESEARCH AND TECHNOLOGY CORPORATION, Indianapolis, IN (US)

(21) Appl. No.: 15/316,833

(22) PCT Filed: Jun. 10, 2015
(86) PCT No.: PCT/US15/35152

§ 371 Date: Dec. 6, 2016

Related U.S. Application Data
(60) Provisional application No. 62/010,309, filed on Jun. 10, 2014.

(51) Int. Cl. A61B 10/02; A61B 17/3211; A61B 17/34; A61B 17/3205

(52) U.S. Cl. CPC A61B 10/0266; A61B 17/3205; A61B 17/3211; A61B 17/34; A61B 2017/320052

(57) ABSTRACT

A method and apparatus for extracting tissue samples are disclosed herein. An example apparatus disclosed herein includes a first sidewall and a second sidewall spaced apart from the first sidewall. The example apparatus also includes a track to receive a blade between the first sidewall and the second sidewall. The track is to guide the blade to penetrate skin of a patient to a predetermined depth of invasion.

### CROSS-REFERENCE TO RELATED APPLICATIONS

This application claims the benefit of U.S. Provisional Patent Application No. 62/010,309, which was filed on Jun. 10, 2014 and is incorporated by reference herein in its entirety.

### STATEMENT REGARDING FEDERALLY SPONSORED RESEARCH

N/A

### BACKGROUND OF THE DISCLOSURE

The field of the disclosure is related to biopsies. More particularly, the disclosure relates to systems and methods for extracting tissue samples.

Skin cancer is the most common human cancer. For example, some 2 million diagnoses of skin cancer were made in 2010 alone. Melanoma is the most lethal skin cancer and third most common skin cancer. Furthermore, melanoma incidence is rapidly rising with a 600% increase from 1950 to 2000. Prognosis of malignant melanoma is directly related to the depth of tumor invasion. Clinical management is dependent on and guided by an accurate depth of invasion as assessed by biopsy.

Generally, a skin cancer diagnosis is made following results of a skin biopsy. The most common biopsy procedure is a shave biopsy procedure. The shave biopsy procedure typically involves sliding a razor blade back and forth by hand to remove a tissue sample from a patient. Advantageously, shave biopsies require few stitches when compared to alternative techniques. Unfortunately, traditional shave techniques are hampered by an inability to ensure an accurate biopsy depth. As a result, additional biopsy procedure or a surgery may be needed because of the inadequacy of the initial shave biopsy. This increases healthcare expenditures and delays timely definitive therapy for the melanoma patient. For example, when a skin lesion is biopsied using a shave biopsy technique and is of insufficient depth, the pathological analysis will identify malignant melanoma with a positive deep margin. This translates to cancer left behind and an unknown depth of invasion.

Presently, the shave biopsy technique fails to adequately quantify an accurate depth of tissue harvest for biopsy. For example, simplistically, critical decision points in melanoma management involve a depth of invasion of 1 mm or greater. A greater than 1 mm depth of invasion implies the need for a larger area of skin resection, as well as lymph node basin sampling. As a result, current medical/surgical dogma dictates that the clinician use a punch biopsy to study any suspicious lesion. This is because the punch biopsy provides full depth information for future intervention. Unfortunately, the relative invasiveness and requisite stitches cause many patients and clinicians to favor the traditional shave biopsy, despite the above-described drawbacks of the shave biopsy.

Therefore, it would be advantageous to have a system and method for performing biopsies that is capable of securing tissue samples that provide necessary clinical and pathological information, while reducing the invasiveness and/or cosmetic impact on the patient.

### SUMMARY OF THE DISCLOSURE

The present disclosure provides a system and method for securing tissue samples from a subject that enables the clinician to accurately quantify the biopsy depth during a biopsy procedure, including a shave biopsy procedure. In particular, a biopsy device is provided that includes complementary mechanical structures that cooperate to control the biopsy procedure and provide feedback to the operator to, thereby, quantify the biopsy depth.

An example apparatus disclosed herein includes a first sidewall and a second sidewall spaced apart from the first sidewall. The example apparatus also includes a track to receive a blade between the first sidewall and the second sidewall. The track is to guide the blade to penetrate skin of a patient to a predetermined depth of invasion.

Another example apparatus disclosed herein includes a fixture including an anchor. The anchor is to penetrate skin of a patient. The example apparatus also includes a blade to contact a portion of the anchor to sever a tissue sample from the patient.

Another example apparatus disclosed herein includes a fixture including an anchor. The anchor is to secure the fixture to skin of a patient. The example apparatus also includes a blade to cooperate with the anchor to sever a tissue sample from the patient at a predetermined depth of invasion.

The foregoing and other aspects and advantages of the examples disclosed herein will appear from the following description. In the description, reference is made to the accompanying drawings which form a part hereof, and in which there is shown by way of illustration example apparatus to extract tissue samples disclosed herein.

### BRIEF DESCRIPTION OF THE DRAWINGS

FIG. 1 is a top view of an example shave biopsy blade disclosed herein.

FIG. 2 is a top view of another example shave biopsy blade disclosed herein.

FIG. 3 is front view of an example fixture disclosed herein, which may be used to guide the example shave biopsy blade of FIG. 1 and/or the example shave biopsy blade of FIG. 2 into and through skin of a patient.

FIG. 4 is a perspective view of a track of the example fixture of FIG. 3.

FIG. 5 is a side view of the example track of FIG. 4.

FIG. 6 is cross-sectional view of an example shave biopsy blade received in the example track of the fixture of FIGS. 3-5.

FIG. 7 is a top view of another example shave biopsy blade disclosed herein.

FIG. 8 is a perspective view of another example fixture and example shave biopsy blade in accordance with the teachings of this disclosure.

FIG. 9 is a side view of the example fixture and the example shave biopsy blade of FIG. 8.

FIG. 10 is another perspective view of the example fixture of FIGS. 8-9.

FIG. 11 is a cross-sectional view of the example fixture of FIGS. 9-10 taken along line 11-11.

### DETAILED DESCRIPTION

System and method to extract tissue samples are disclosed herein. An example apparatus disclosed herein includes a fixture to be anchored to skin of a patient. In some examples, the fixture includes a track that is to receive a shave biopsy blade. The track guides the shave biopsy blade into the skin of the patient to a predetermined depth of invasion. As a result, the example apparatus disclosed herein accurately and consistently sever tissue samples at the predetermined depth of invasion, thereby facilitating diagnosis and clinical management of medical conditions (e.g., skin cancers such as melanoma).

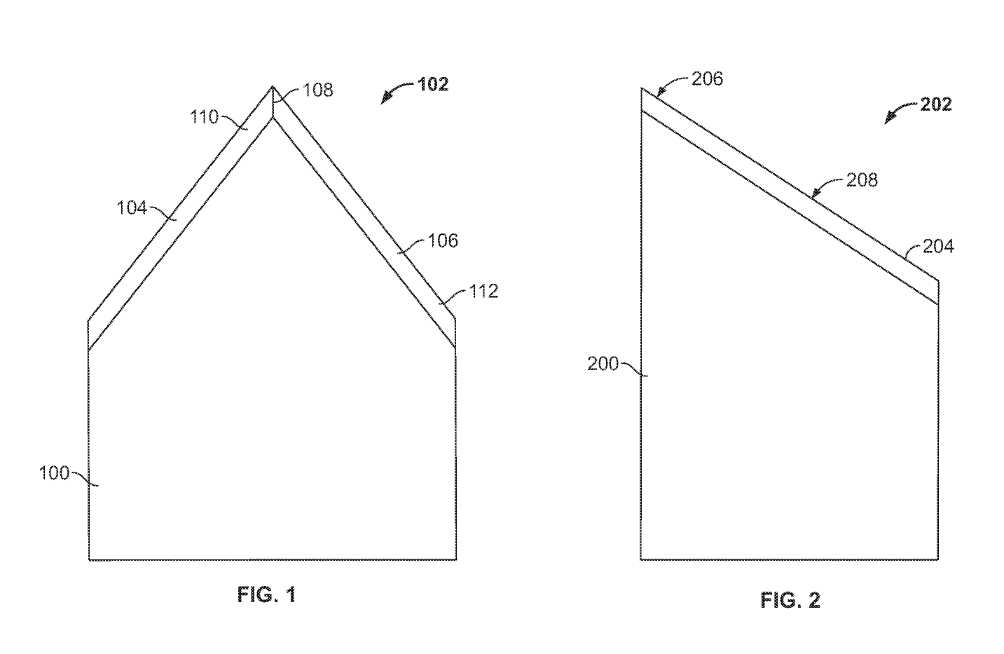

FIG. 1 is a top view of a head 100 of an example shave biopsy blade 102 disclosed herein. In the illustrated example, the head 100 of the example shave biopsy blade 102 is triangular or arrowhead shaped. The example head 100 of the example shave biopsy blade 102 has a first cutting edge 104 and a second cutting edge 106. The example second cutting edge 106 is oriented at an angle relative to the first cutting edge 104. In the illustrated example, the first cutting edge 104 is oriented at a sixty degree angle relative to the second cutting edge 106. However, the above-noted angle is merely an example and, thus, the first cutting edge 104 may be oriented at other angles relative to the second cutting edge 106 (e.g., 100 degrees, 130 degrees, 150 degrees, 165 degrees, etc.) As a result, the cutting edges 104, 106 of the example shave biopsy blade 102 include a leading portion 108 and respective trailing portions 110, 112. In the illustrated example, the leading portion 108 is a pointed tip, which facilitates piercing, penetration and/or cutting of skin of a patient. As a result, the example shave biopsy blade 102 may be moved (e.g., pressed or pushed) in only one direction through the skin of the patient to sever a tissue sample from the skin of the patient.

FIG. 2 is a top view of a head 200 of another example shave biopsy blade 202 in accordance with the teachings of this disclosure. In the illustrated example, the head 200 of the shave biopsy blade 202 is right-triangular shaped and includes a substantially planar or straight cutting edge 204. The example cutting edge 204 of FIG. 2 is to be oriented at an angle relative to the skin of the patient such that a first or leading portion 206 of the cutting edge 204 initially contacts and pierces the skin, and a second or trailing portion 208 of the cutting edge 206 further cuts and/or penetrates the skin as the shave biopsy blade 202 is driven (e.g., pressed) into the skin. Thus, the shape of the example shave biopsy blade 202 facilitates piercing, penetration and/or cutting of the skin of the patient. As a result, the example shave biopsy blade 202 may be moved (e.g., pressed or pushed) in only one direction through the skin of the patient to sever a tissue sample from the skin of the patient. The above-noted shapes of the heads 100, 200 of the shave biopsy blades 102, 202 of FIGS. 1 and 2, respectively, are merely examples and, thus, the shave biopsy blades 102, 202 may be implemented using other shapes without departing from the scope of this disclosure. For example, the shave biopsy blades 102, 202 may be implemented with rounded or curved cutting edges.

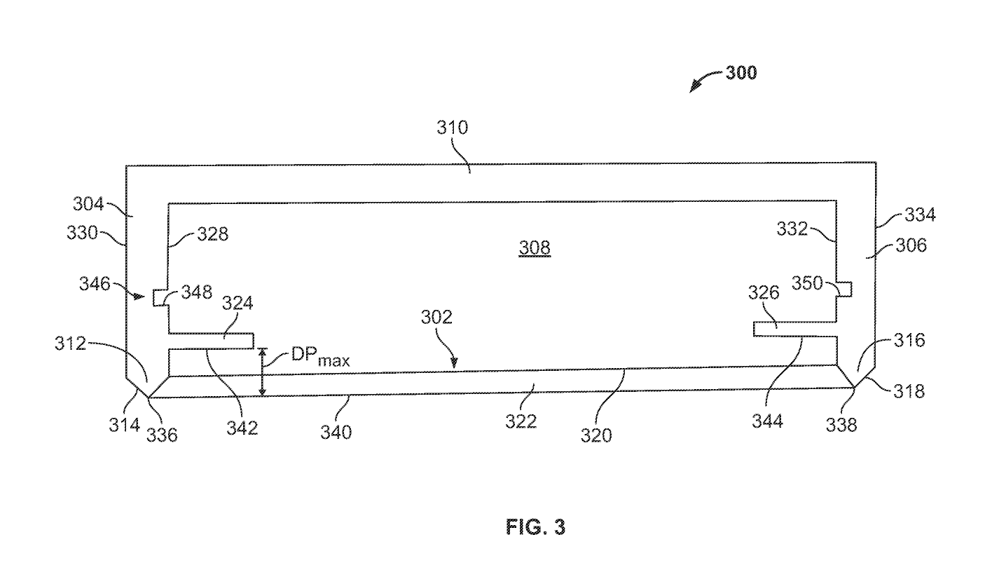

FIG. 3 is a front view of an example of a fixture 300 disclosed herein that may be used to support and guide a shave biopsy blade (e.g., the shave biopsy blade 102 of FIG. 1, the shave biopsy blade 202 of FIG. 2, and/or any other suitable shave biopsy blade) to extract or harvest a tissue sample from the skin of the patient. In the illustrated example, the fixture 300 defines a portion of a chamber 302 to capture the tissue sample. The example fixture 300 includes a first sidewall 304, a second sidewall 306, an end wall 308 and a top wall 310. In some examples, two or more of the first sidewall 304, the second sidewall 306, the end wall 308 and/or the top wall 310 are integrally formed. In some examples, two or more of the first sidewall 304, the second sidewall 306, the end wall 308 and/or the top wall 310 are coupled to each other via one or more mechanical fasteners and/or connections (e.g., adhesives, welds, rivets, bolts, screws, nails, clamps, press-fit connections and/or one or more additional and/or alternative mechanical fasteners and/or connections).

In the illustrated example, the first sidewall 304 is substantially parallel and contralateral to the second sidewall 306. The example top wall 310 of the example fixture 300 is substantially perpendicular to the example first sidewall 304 and the example second sidewall 306. In some examples, the shave biopsy blade is to contact the end wall 308 to sever the tissue sample from the patient. Thus, in some examples, the end wall 308 is shaped and/or oriented at an angle relative to the first sidewall 304 and the second sidewall 306 that corresponds to a shape and/or an orientation of a cutting edge of the shave biopsy blade. For example, if the shave biopsy blade 102 of FIG. 1 is employed, the end wall 308 may have a V-shaped cross-sectional profile or shape corresponding to the triangular shape of the head 100 to enable the leading portion 108 and the trailing portions 110, 112 of the cutting edge 104 to contact the end wall 308. If the shave biopsy blade 202 of FIG. 2 is employed, for example, the end wall 308 may be oriented at an angle relative to the first sidewall 304 and the second sidewall 306 corresponding to the angle at which the cutting edge 204 is oriented to enable the leading portion 206 and the trailing portion 208 of the cutting edge 204 to contact the end wall 308. In other examples, the first sidewall 304, the second sidewall 306, the end wall 308, and/or the top wall 310 are oriented and/or configured in other ways. In the illustrated example, the first sidewall 304, the second sidewall 306, the end wall 308, and the top wall 310 are substantially straight or planar. In other examples, the first sidewall 304, the second sidewall 306, the end wall 308, and/or the top wall 310 are configured in other ways. For example, the first sidewall 304, the second sidewall 306, the end wall 308, and/or the top wall 310 may be curved.

In the illustrated example, a first distal end 312 of the first sidewall 304 includes a first blade 314. A second distal end 316 of the example second sidewall 306 includes a second blade 318. A third distal end 320 of the example end wall 308 includes a third blade 322. In the illustrated example, the first blade 314, the second blade 318, and the third blade 322 are defined by the first sidewall 304, the second sidewall 306, and the end wall 308, respectively. In some examples, the first blade 314, the second blade 318, and/or the third blade 322 are coupled to the first sidewall 304, the second sidewall 306, and/or the end wall 308, respectively. In the illustrated example, the first blade 314, the second blade 318, and the third blade 322 have V-shaped cross-sectional profiles. In other examples, the first blade 314, the second blade 318, and/or the third blade 322 have cross-sectional profiles of other shapes. In some examples, the first blade 314, the second blade 318, and/or the third blade 322 comprise a plurality of blades, pins, teeth, and/or one or more additional and/or alternative components to pierce and/or penetrate the skin of the patient.

The example first blade 314, the example second blade 318, and the example third blade 322 penetrate the skin of the patient to anchor or secure the fixture 300 to the skin. For example, the first blade 314, the example second blade 318, and the example third blade 322 substantially vertically incise the skin of the patient. In some examples, the first blade 314, the second blade 318 and/or the third blade 322 cut portions of the skin to define a perimeter of a tissue sample to be extracted from the patient. For example, a clinician or technician may identify a skin lesion visible on the skin of the patient and place the fixture 300 on top of and/or around a portion of the lesion and/or skin that is to be extracted from the patient. In some examples, the fixture 300 is placed over the lesion such that the first sidewall 304, the second sidewall 306 and the end wall 308 frame or surround the portion of the skin lesion that is to be extracted. In some examples, to facilitate positioning of the fixture 300 relative to the skin lesion, the top wall 310 includes one or more openings, windows, and/or transparent portions to enable the clinician or technician to view the skin and/or the skin lesion through the top wall 310 when the fixture 300 is positioned on the skin and/or as the fixture 300 is being placed (e.g., lowered) onto the skin.

The clinician or technician may then press the fixture 300 towards the skin (e.g., downward in the orientation of FIG. 3) with force sufficient to pierce the skin via the first blade 314, the second blade 318, and the third blade 322. In the illustrated example, because the end wall 308 is non-parallel (e.g., substantially perpendicular) to the first sidewall 304 and the second sidewall 306, the first sidewall 304, the second sidewall 306, and the end wall 308 cooperate to resist and/or prevent lateral and/or longitudinal movement (e.g., sliding) of the fixture 300 along the skin of the patient as the first blade 314, the second blade 318, and the third blade 322 pierce and penetrate the skin. This, in the illustrated example, the first blade 314, the second blade 318, and the third blade 322 cooperate to be an anchor of the fixture 300 and facilitate accurate placement of the fixture 300 relative to the lesion.

In the illustrated example, the fixture 300 includes a first stop 324 and a second stop 326 to limit a depth at which the first blade 314, the second blade 318, and the third blade 322 penetrate the skin of the patient. The example first stop 324 of FIG. 3 is a protrusion or tab extending from a first interior side 328 of the first sidewall 304 toward the second sidewall 306. In some examples, the first stop 324 extends from a first exterior side 330 of the first sidewall 304. The example second stop 326 of FIG. 3 is a protrusion or tab extending from a second interior side 332 of the second sidewall 306 toward the first sidewall 304. In some examples, the second stop 326 extends from a second exterior side 334 of the second sidewall 306. Although the example fixture 300 of FIG. 3 includes two stops (the first stop 324 and the second stop 326), other examples may include other numbers of stops (e.g., 1, 3, 4, 5, 6, 7, etc.). For example, in some examples, the fixture 300 includes a third stop extending from the end wall 308. In the illustrated example, the first stop 324 and the second stop 326 have rectangular cross-sectional profiles. In other examples, the first stop 324 and/or the second stop 326 have cross-sectional profiles having other shapes.

As mentioned above, the example first stop 324 and the example second stop 326 limit a depth of penetration of the first blade 314, the second blade 318, and the third blade 322 into the skin of the patient. For example, the first stop 324 and the second stop 326 of FIG. 3 may contact a surface of the skin of the patient as the clinician or technician presses the first blade 314, the second blade 318, and the third blade 322 of the fixture 300 into the skin of the patient. As a result, the first stop 324 and the second stop 326 substantially prevent further penetration of the first blade 314, the second blade 318, and the third blade 322 into the skin. In some examples, when the first stop 324 and/or the second stop 326 contact the skin, the fixture 300 provides tactile feedback to the clinician or technician to indicate that the first blade 314, the second blade 318, and the third blade 322 penetrated the skin to a maximum depth of penetration DPₘₐₓ.

In the illustrated example, the maximum depth of penetration DPₘₐₓ of the skin by the first blade 314, the second blade 318, and the third blade 322 corresponds to a distance or depth from cutting edges 336, 338, 340 of the first blade 314, the second blade 318, and the third blade 322, respectively, to contacting surfaces 342, 344 (e.g., a lower or bottom surface) of the first stop 324 and the second stop 326, respectively. In the illustrated example, the maximum depths of penetration DPₘₐₓ of the first blade 314, the second blade 318, and the third blade 322 are substantially the same. In other examples, the maximum depths of penetration DPₘₐₓ of the first blade 314, the second blade 318, and/or the third blade 322 are different. For example, the end wall 308 and/or the third blade 322 may be dimensioned such that the third blade 322 penetrates deeper into the skin than the first blade 314 and/or the second blade 318 when the first stop 324 and the second stop 326 contact the skin.

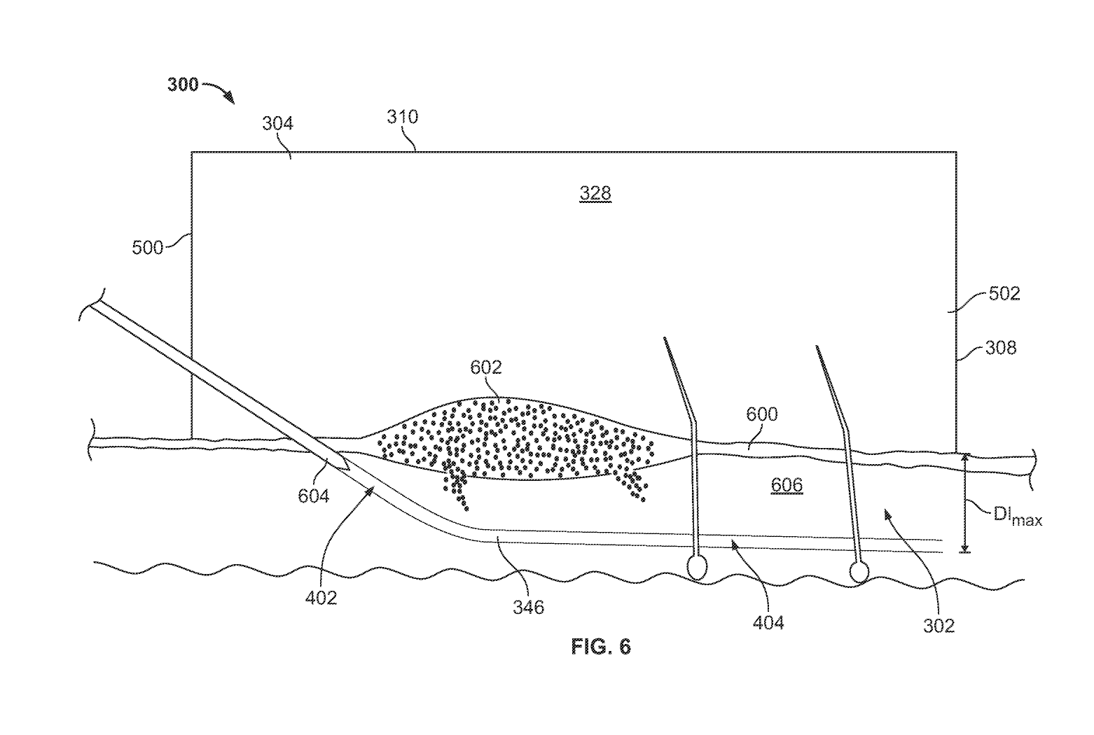

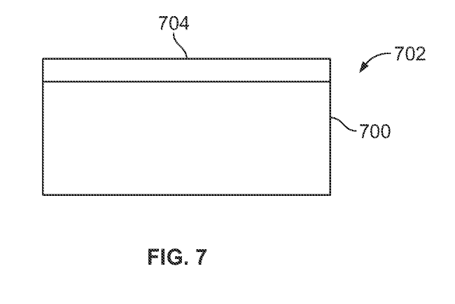

The example fixture 300 of FIG. 3 includes a track 346 to receive a shave biopsy blade 604 between the first sidewall 304 and the second sidewall 306 as, for example, illustrated in FIG. 6. As the shave biopsy blade 604 moves along the track 346, the track 346 guides the shave biopsy blade 604 into the skin and along a predetermined depth of invasion DIₘₐₓ (FIG. 6). As a result, the shave biopsy blade 604 laterally and/or horizontally incises the skin of the patient. As described with reference to FIG. 6 below, the shave biopsy blade 604 may be implemented by the shave biopsy blade 102 of FIG. 1, the shave biopsy blade 202 of FIG. 2, the shave biopsy blade 702 of FIG. 7 and/or any other suitable shave biopsy blade. In the illustrated example, the track 346 includes a first channel or slot 348 defined by the first side wall 304. The example track 346 of FIG. 3 also includes a second channel or slot 350 defined by the second sidewall 306. In other examples, the track 346 may be implemented by other structures such as, for example, protrusions defining the first channel 348 and/or the second channel 350.

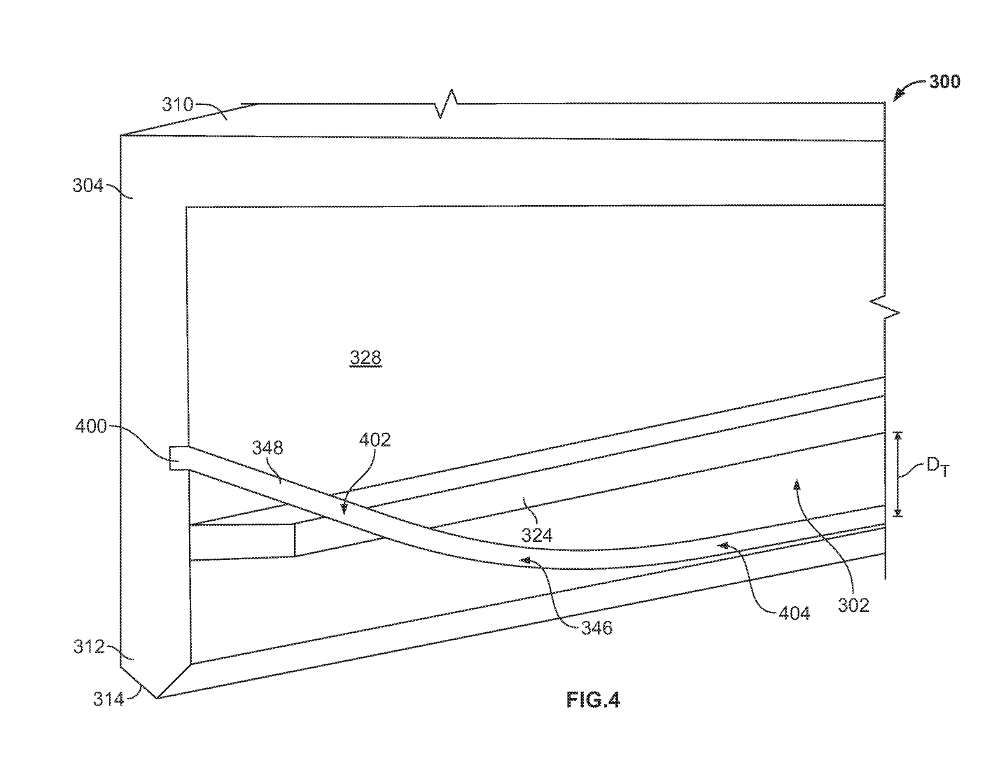

FIG. 4 is a perspective view of the example track 346. In the illustrated example, a first end 400 of the track 346 is disposed above the first stop 324 in the orientation of FIG. 4. Thus, when the fixture 300 is anchored in the skin of the patient, the first end 400 of the track 346 is disposed above the skin and is accessible to receive the shave biopsy blade 604. A first portion 402 of the example track 346 extends away from the top wall 310. In the illustrated example, the first portion 402 of the track 346 extends from above the first stop 324 to below the first stop 324. Thus, a second portion 404 of the example track 346 is disposed below the first stop 324. In some examples, the first portion 402 of the track 346 is substantially straight or linear. In other examples, the first portion 402 of the track 346 is curved or rounded. In some examples, the first portion 402 of the track 346 has one or more substantially linear sections and one or more curved sections. In the illustrated example, the second portion 404 of the track 346 is substantially parallel to the first contacting surface 342 of the first stop 324 and is spaced apart from the first contacting surface 342 of the first stop 324 by a distance D_{T}. As a result, when the fixture 300 is anchored in the skin of the patient and the shave biopsy blade 604 is inserted into the track 346 and moved along the track 346, the shave biopsy blade 604 penetrates the skin and cuts or shaves the skin at the predetermined depth of invasion DIₘₐₓ.

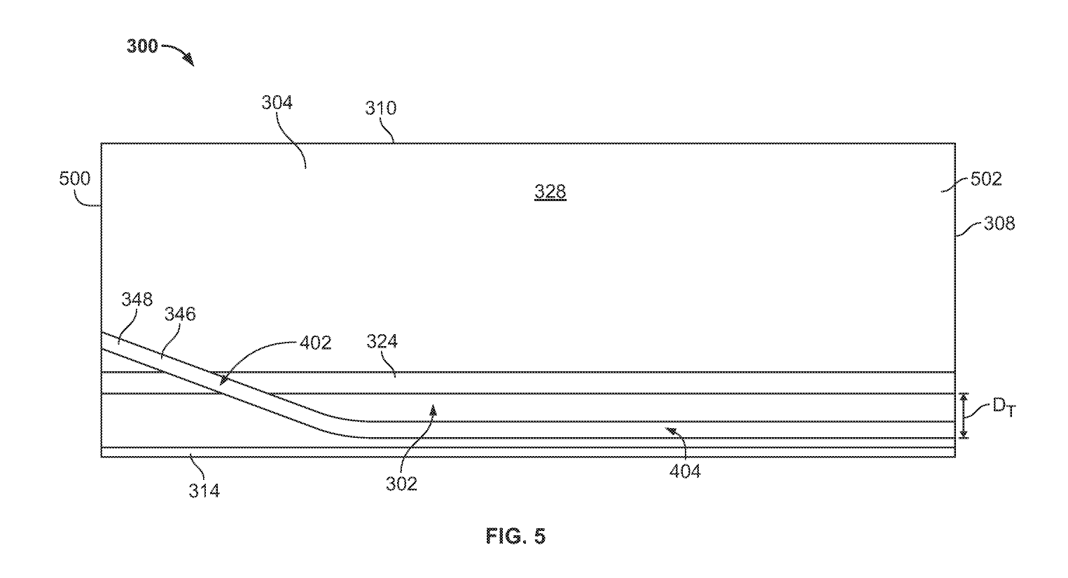

FIG. 5 is a side view of the example track 346. In the illustrated example, the stops 324, 326 and the track 346 extend from a first end 500 of the fixture 300 to a second end 502 of the fixture 300. In other examples, the stops 324, 326 and/or the track 346 do not extend from the first end 500 to the second end 502 of the fixture 300. For example, the stops 324, 326 may be spaced apart from the first end 500 and/or the second end 502.

FIG. 6 is a schematic side view of the example fixture 300 anchored to skin 600 of a patient. In the illustrated example, the fixture 300 is employed to extract a tissue sample including a skin lesion 602. For example, in the illustrated example, the first blade 314, the second blade 318, and the third blade 322 of the first sidewall 304, the second sidewall 306, and the end wall 308, respectively, penetrate the skin 600 to a depth at which the stops 324, 326 contact the skin 600. An example shave biopsy blade 604 (e.g., the shave biopsy blade 102 of FIG. 1, the shave biopsy blade 202 of FIG. 2, and/or any other suitable shave biopsy blade) is received in the track 346 and moves along a path defined by the track 346. As a result, the example shave biopsy blade 604 is guided into the skin 600 fore of the skin lesion 602. As the shave biopsy blade 604 moves through the second portion 404 of the track 346, the shave biopsy blade 604 penetrates the skin at the predetermined depth of invasion DI_{max,} and then contacts the end wall 308. In the illustrated example, the first blade 314, the second blade 318, and the third blade 322 penetrate the skin deeper than the predetermined depth of invasion DIₘₐₓ.

When the example shave biopsy blade 604 contacts the end wall 308, the first blade 314, the second blade 318, the third blade 322, and the shave biopsy blade 604 cooperate to sever a tissue sample 606 from the skin 600 and capture the tissue sample 606 in the chamber 302. In some examples, the tissue sample 606 has a predetermined thickness corresponding to the predetermined depth of invasion Dlₘₐₓ. In some examples, to extract the tissue sample 606 from the patient, the fixture 300 and the shave biopsy blade 604 are lifted together from the skin 600. In other examples, the shave biopsy blade 604 is withdrawn from the fixture 300, the fixture 300 is lifted from the skin 606, and the tissue sample 606 is extracted via a tool (e.g., tweezers) and/or by hand. In some examples, the fixture 300 does not include the end wall 308, and the tissue sample is severed by cutting a portion of the skin adjacent the second end 502 of the fixture with, for example, a scalpel or scissors.

FIG. 7 is a top view of a head 700 of another example shave biopsy blade 702 disclosed herein, which may be used to implement the example shave biopsy blade 604 of FIG. 6. In the illustrated example, the head 700 is rectangular-shaped and includes a substantially planar or straight cutting edge 704. The example shave biopsy blade 702 may be moved (e.g., pressed or pushed) in only one direction through the skin of the patient to sever a tissue sample from the skin of the patient. Other shave biopsy blades may have heads of other shapes and/or different types of cutting edges (e.g., angled, pointed, etc.).

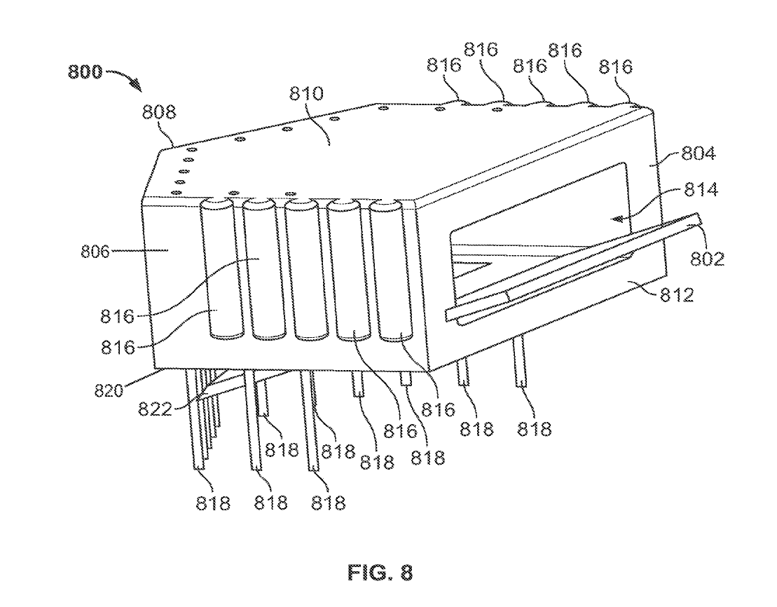

FIG. 8 is a front, perspective view of an another example fixture 800 disclosed herein, which may be used to support and guide a shave biopsy blade 802 (e.g., the shave biopsy blade 102 of FIG. 1, the shave biopsy blade 202 of FIG. 2, the shave biopsy blade 702 of FIG. 7 and/or any other suitable shave biopsy blade) to extract or harvest a tissue sample from the skin of the patient. The example fixture 800 includes a first sidewall 804, a second sidewall 806, a V-shaped end wall 808, a top wall 810, and a bottom wall 812. In some examples, two or more of the first sidewall 804, the second sidewall 806, the end wall 808, the top wall 810, and/or the bottom wall 802 are integrally formed. In some examples, two or more of the first sidewall 804, the second sidewall 806, the end wall 808, the top wall 810, and/or the bottom wall 812 are coupled to each other via one or more mechanical fasteners and/or connections (e.g., adhesives, welds, rivets, bolts, screws, nails, clamps, press-fit connections and/or one or more additional and/or alternative mechanical fasteners and/or connections). Although the example end wall 808 of FIG. 8 is V-shaped, the end wall 808 may be other shapes (e.g., planar, round, etc.) in other examples.

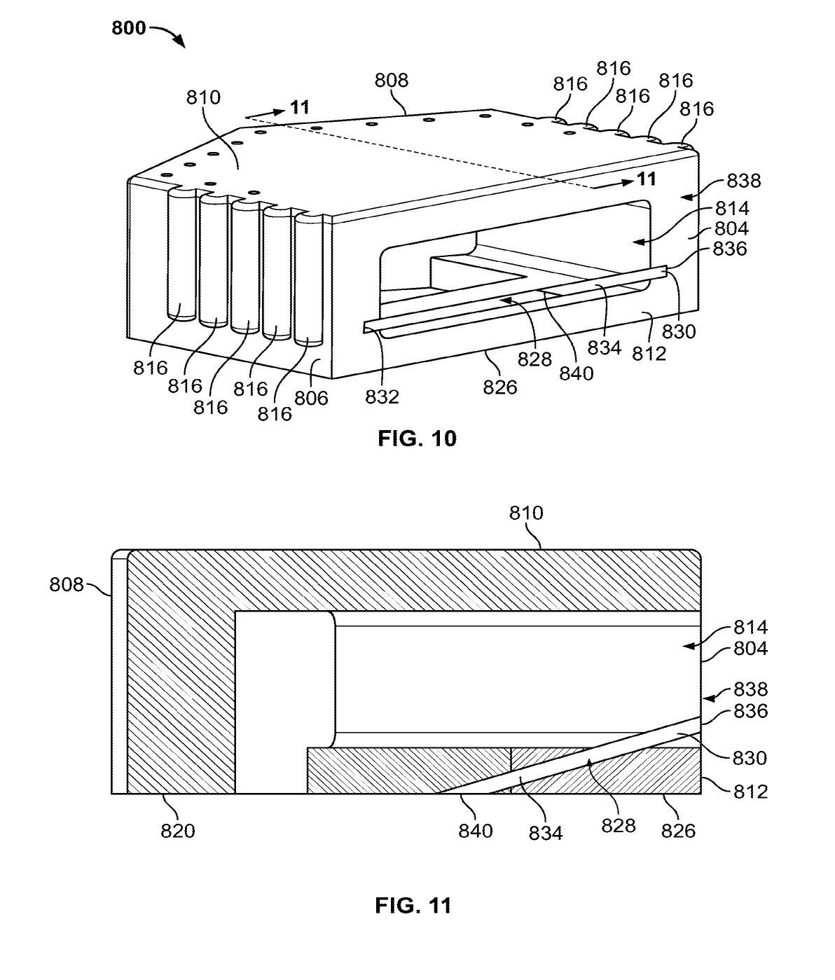

In the illustrated example, the first sidewall 804 is substantially parallel and contralateral to the second sidewall 806. The example top wall 810 is substantially parallel and contralateral to the bottom wall 812. The top wall 810 and the bottom wall 812 of the example fixture 800 are substantially perpendicular to the example first sidewall 804 and the example second sidewall 806. In the illustrated example, the fixture 800 includes an opening 814 contralateral to the end wall 808. As described in greater detail below, the shave biopsy blade 802 is inserted into a track 828 (FIG. 10) of the fixture 800 via the opening 814. In the illustrated example, the first sidewall 804 and the second sidewall 806 include a plurality of protrusions 816 to facilitate gripping and/or holding of the fixture 800. In the illustrated example, the first sidewall 804, the second sidewall 806, sections of the end wall 808, the top wall 810, and the bottom wall 812 are substantially straight or planar. In other examples, the first sidewall 804, the second sidewall 806, the end wall 808, the top wall 810 and/or the bottom wall 812 are configured in other ways. For example, the first sidewall 804, the second sidewall 806, the end wall 808, the top wall 810, and/or the bottom wall 812 may be curved.

The bottom wall 812 rests on and/or contacts skin of a patient when a tissue sample is extracted via the blade 802. In the illustrated example, a plurality of pins or needles 816 (e.g., 30 gauge needles) are coupled to the fixture 800 and project from the bottom wall 812. The needles 818 of FIG. 8 project substantially perpendicularly to the bottom wall 812 to enable the needles 818 to pierce the skin of the patient and secure the fixture 802 to the patient. Thus, the needles 818 cooperate to be an anchor that penetrates the skin of the patient and substantially prevents lateral and/or longitudinal movement (e.g., sliding) of the fixture 800 along the skin of the patient.

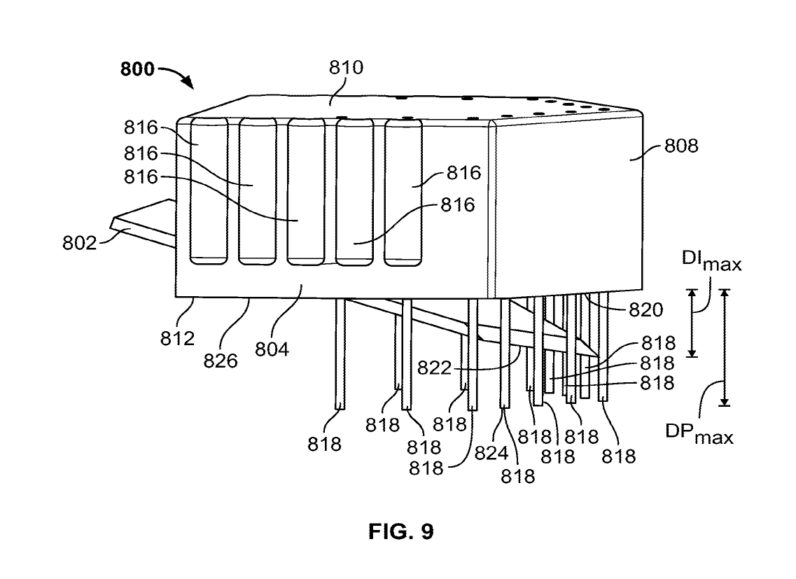

FIG. 9 is a side view of the example fixture 800 of FIG. 8. In some examples, the shave biopsy blade 802 is to contact the needles 818 to sever the tissue sample from the patient. Thus, in some examples, the needles 818 are positioned such that the needles 818 form a pattern that corresponds to a shape and/or an orientation of a cutting edge 822 of the shave biopsy blade 802 to enable the shave biopsy blade 802 and the needles 818 to cooperate to sever the tissue sample from the patient. For example, the needles 818 may be positioned in a V-shaped pattern corresponding to V-shaped cutting edge 822 of the shave biopsy blade 802 to enable the cutting edge 822 to contact the needles 818 at a plurality of points along the cutting edge 822. In some examples, the cutting edge 822 of the shave biopsy blade 802 has other shapes and/or another shave biopsy blade is employed. In such examples, the needles 818 may be positioned to form other patterns. For example, if the shave biopsy blade 202 of FIG. 2 is employed, the needles 818 may be positioned in a line at an angle relative to the first sidewall 804 and the second sidewall 806 corresponding to the angle at which the cutting edge 204 is oriented to enable the leading portion 206 and the trailing portion 208 of the cutting edge 204 to contact the needles 818. In some examples, the needles 818 are positioned along or near a border 820 of the bottom wall 812.

During a biopsy procedure, a clinician or technician may identify a skin lesion visible on the skin of the patient and place the fixture 800 on top of and/or around a portion of the lesion and/or skin that is to be extracted from the patient. In some examples, the fixture 800 is placed over the lesion such that the first sidewall 804, the second sidewall 806 and the end wall 808 frame or surround the portion of the skin lesion that is to be extracted. In some examples, to facilitate positioning of the fixture 800 relative to the skin lesion, the top wall 810 and/or the bottom wall 812 includes one or more openings, windows, and/or transparent portions to enable the clinician or technician to view the skin and/or the skin lesion through the top wall 810 and/or the bottom wall 812 when the fixture 800 is positioned on the skin and/or as the fixture 800 is being placed (e.g., lowered) onto the skin. The clinician or technician may then press the fixture 800 towards the skin (e.g., downward in the orientation of FIG. 9) with force sufficient to pierce the skin via the needles 818.

The example bottom wall 812 limits a depth of penetration of the needles 818 into the skin of the patient. For example, the bottom wall 812 may contact a surface of the skin of the patient as the clinician or technician presses the needles 818 of the fixture 800 into the skin of the patient. As a result, the bottom wall 812 is a stop that substantially prevents further penetration of the needles 818 into the skin. In some examples, when the bottom wall 812 contacts the skin, the fixture 800 provides tactile feedback to the clinician or technician to indicate that the needles 818 penetrated the skin to a maximum depth of penetration DPₘₐₓ. In the illustrated example, the maximum depth of penetration DPₘₐₓ of the skin by the needles 818 corresponds to a distance or depth from tips 824 of the needles 818 to a contacting surface 826 (e.g., a lower or bottom surface) of the bottom wall 812. In the illustrated example, the maximum depth of penetration DPₘₐₓ of each of the needles 818 is substantially the same. In other examples, the maximum depths of penetration DPₘₐₓ of the needles 818 are different. For example, some of the needles 818 may project farther from the bottom wall 812 than other ones of the needles 818.

When the shave biopsy blade 802 is inserted into the fixture 800, the shave biopsy blade 802 moves into the skin to a predetermined depth of invasion DIₘₐₓ. As a result, the shave biopsy blade 802 incises the skin of the patient. In the illustrated example, when the shave biopsy blade 802 is at the predetermined depth of invasion DIₘₐₓ, the cutting edge 822 contacts the needles 818 to sever a tissue sample of a predetermined shape (e.g., wedge-shaped, block-shaped, and/or any other desired shape) and a predetermined size from the patient. In some examples, the predetermined depth of invasion DIₘₐₓ is between about 1 millimeter and about 2 millimeters. In other examples, the predetermined depth of invasion DIₘₐₓ is other depths (e.g., between about 0.5 millimeters and about 2.5 millimeters, between about 1.1 millimeter and about 1.5 millimeters, and/or any other suitable depth(s).) In the illustrated example, the predetermined depth of invasion DIₘₐₓ of the shave biopsy blade 802 is less deep than the maximum depths of penetration DPₘₐₓ of the needles 818. Thus, the needles 818 may penetrate deeper into the skin of the patient than the shave biopsy blade 802. In some examples, the predetermined depth of invasion DIₘₐₓ of the shave biopsy blade 802 is substantially the same depth as the maximum depth of penetration DPₘₐₓ of one or more of the needles 818

FIG. 10 is a perspective view of the fixture 800 of FIGS. 8-9 illustrated without the shave biopsy blade 802 and the needles 818. The example fixture 800 of FIG. 10 includes a track 828 to receive the shave biopsy blade 802 between the first sidewall 804 and the second sidewall 806 as, for example, illustrated in FIGS. 8 and 9. In the illustrated example, the track 828 includes a first channel or slot 830 defined by the first sidewall 804. The example track 828 of FIG. 10 also includes a second channel or slot 832 defined by the second sidewall 806. The example track 828 further includes a third channel or slot 834 extending through the bottom wall 812. In the illustrated example, the first channel 830 is continuous with the third channel 834. The example second channel 832 is continuous with the third channel 834. In other examples, the track 828 may be implemented in other ways.

In the illustrated example, a first end 836 of the track 828 is disposed above the bottom wall 812 in the orientation of FIG. 10 on a side 838 of the fixture 800 defining the opening 814. Thus, when the fixture 800 is anchored in the skin of the patient via the needles 818, the first end 836 of the track 828 is disposed above the skin and is accessible to receive the shave biopsy blade 802. A second end 840 of the track 828 is defined by the contacting surface 826 of the bottom wall 812. As a result, when the fixture 800 is anchored in the skin of the patient via the needles 818 and the shave biopsy blade 802 is inserted into the track 828 and moved along the track 828, the track 828 guides the shave biopsy blade 802 to penetrate the skin and sever a tissue sample when the cutting edge 822 reaches the predetermined depth of invasion DIₘₐₓ.

FIG. 11 is a cross-sectional view of the fixture 800 along line 11-11 of FIG. 10. In the illustrated example, the track 828 is substantially straight or linear and declines from the first end 836 to the second end 840. As a result, the track 828 guides the shave biopsy blade 802 in a path that is a substantially straight line when the shave biopsy blade 802 is inserted into the track and moved into contact with the needles 818. As a result, the shave biopsy blade 802 cooperates with the needles 818 to sever a wedge-shaped tissue sample from the patient. In other examples, the shave biopsy blade 802 cooperates with the needles 818 to sever a tissue sample having a different shape. In some examples, when the shave biopsy blade 802 and the needles 818 cooperate to sever the tissue sample from the skin of the patient, the shave biopsy blade 802 captures the tissue sample between the shave biopsy blade 802 and the contacting surface 826 of the bottom wall 812. In some examples, to extract the tissue sample from the patient, the fixture 800 and the shave biopsy blade 802 are lifted together from the skin of the patient. In other examples, the shave biopsy blade 802 is withdrawn from the fixture 800, the fixture 800 is lifted from the skin, and the tissue sample is extracted via a tool (e.g., tweezers) and/or by hand.

The example methods, apparatus, and systems disclosed herein enable tissue samples to be accurately and consistently extracted at a predetermined depth of invasion. Thus, the examples disclosed herein reduce and/or eliminate a risk of extracting tissue samples at insufficient depths and/or at unknown and/or inconsistent depths. As a result, the examples disclosed herein decrease and/or eliminate a risk of having to perform additional biopsy procedures and/or surgeries to extract a tissue sample at a desired depth. In addition, because the examples disclosed herein employ tracks to guide a shave biopsy blade, extracting tissue samples via the examples disclosed herein is simpler, quicker, and more reliable and accurate than extracting tissue samples using traditional shave biopsy techniques.

Although some example apparatus have been disclosed herein, many equivalents, alternatives, variations, and/or modifications, aside from those expressly stated, are employed in other examples and are within the scope of the present disclosure.

1. An apparatus, comprising:
a first sidewall;
a second sidewall spaced apart from the first sidewall; and
a track to receive a blade between the first sidewall and the second sidewall, the track to guide the blade to penetrate skin of a patient to a predetermined depth of invasion.

2. The apparatus of claim 1 further comprising an anchor to penetrate the skin of the patient.

3. The apparatus of claim 2 further comprising a stop to limit a depth of penetration of the anchor.

4. The apparatus of claim 2, wherein the anchor comprises a needle.

5. The apparatus of claim 2, wherein the anchor comprises a plurality of needles positioned in a pattern corresponding to a shape of a cutting edge of the blade.

6. The apparatus of claim of claim 2, wherein the track is to guide the blade into contact with the anchor.

7. The apparatus of claim 1, wherein the predetermined depth of invasion is between about 1 millimeter and about 2 millimeters.

8. The apparatus of claim 1, further comprising a bottom wall, wherein the track extends through the bottom wall.

9. An apparatus, comprising:
a fixture including an anchor, the anchor to penetrate skin of a patient; and
a blade to contact a portion of the anchor to sever a tissue sample from the patient.

10. The apparatus of claim 9 wherein the fixture includes a track to receive the blade.

11. The apparatus of claim 10, wherein the track is to guide the blade in a substantially straight line.

12. The apparatus of claim 10, wherein the track is defined by a first channel in a first sidewall of the fixture and a second channel in a second sidewall of the fixture.

13. The apparatus of claim 9, wherein the anchor comprises a plurality of needles.

14. The apparatus of claim 9, wherein the blade has a V-shaped cutting edge.

15. The apparatus of claim 9, wherein the fixture comprises a stop to limit a depth at which the anchor is to penetrate the skin of the patient.

16. An apparatus, comprising:
a fixture including an anchor, the anchor to secure the fixture to skin of a patient; and
a blade to cooperate with the anchor to sever a tissue sample from the patient at a predetermined depth of invasion.

17. The apparatus of claim 16, wherein the fixture includes a first sidewall, a second sidewall, and a bottom wall extending between the first sidewall and the second sidewall.

18. The apparatus of claim 17, wherein the first sidewall includes a first channel, the second sidewall includes a second channel, and the bottom wall includes a third channel, and wherein the first channel, the second channel, and the third channel define a track to guide the blade.

19. The apparatus of claim 18, wherein the first channel is continuous with the third channel.

20. The apparatus of claim 16, wherein the anchor is to penetrate deeper into the skin than the blade.
